(19) United States
(12) Patent Application Publication
(10) Pub. No.: US 2009/0112026 A1
(43) Pub. Date: Apr. 30, 2009

(54) Formation of hydroxycarbonyl compounds

(75) Inventor: Melvin Keith Carter, Lincoln, CA (US)

Correspondence Address: M. K. Carter, 2300 Sutter View Lane, Lincoln, CA, 95648, US

(73) Assignee: Carter Technologies, Lincoln, CA (US)

(21) Appl. No.: 11/976,770

(22) Filed: Oct. 29, 2007

(51) Int. Cl. C07C 45/64

(52) U.S. Cl. 568/388; 568/458

(57) ABSTRACT

Aldehyde and ketone reactants have been converted to hydroxyaldehydes, polyhydroxyaldehydes, hydroxyketones and/or polyhydroxyketones in aqueous liquid phase by an aldol condensation process where product carbon chain length was limited using specific dilute concentrations of soluble inorganic base. Reactions were reproducibly conducted at ambient pressure in a temperature range of −25° C. to 50° C. and completed in ten minutes or less following reactant addition.

### BACKGROUND

1. Field of Invention

Aldehydes and ketones have been chemically converted to hydroxyaldehydes, polyhydroxyaldehydes, hydroxyketones and/or polyhydroxyketones by an aldol condensation process where a broad range of products formed in the presence of acids, neutral ionic species and/or base. A number of reactions have been disclosed for formation of reactive molecules of both determined and undetermined carbon-carbon chain length at elevated temperatures, long reaction times and predominantly low conversion efficiencies. The invention taught in this application is chemical conversion of aldehydes and/or ketones to a majority of products of controlled carbon chain length at ambient pressure for reactant contact times of ten minutes or less.

2. Description of Prior Art

The chemical and fuel processing industries have grown to maturity using petroleum feed stocks. Petroleum is a non-renewable or depleting resource that may become unavailable in the next 100 years. This planet Earth fosters continual growth of numerous carbohydrate based plants including fruits, vegetables and grain food sources plus their supporting cellulosic plant stalks and related natural waste materials for recycle. Grains, corn cobs, prairie grasses and other cellulosic materials are, in part, subject to bio-fermentation or thermal processes producing ethanol and related products. A major industry is rapidly developing in ethanol production and much of the product is sold as combustion engine fuel or its additive. Ethanol is becoming more available as a renewable resource and this application teaches conversion of aldehydes, including acetaldehyde derived from ethanol, and ketones to valuable intermediates for use in production of fuels and industrial chemical intermediates.

The invention disclosed in this application teaches rapid chemical conversion of aldehydes and ketones to hydroxyaldehydes, polyhydroxyaldehydes, hydroxyketones and/or polyhydroxyketones in liquid phase using soluble inorganic base. This process provides a method for production of concatenated carbon-carbon backbone reactive molecules of predetermined chain length at ambient pressure for reactant contact times of ten minutes or less. For example, C₂ reactant aldehydes, such as acetaldehyde, are converted to C₄ through Cₙ for n values up to hundreds where the value of n is determined by the concentration of the soluble base. Such concatenated carbon-carbon backbone molecules become a basis for manufacture of hydrocarbon fuels and industrial chemical intermediates by means of a reduction process.

There are a number of hot tube reactions reported for preparation of hydroxyaldehydes, hydroxyketones and/or unsaturated aldehydes conducted at or above ambient pressure by passing aldehyde or ketone vapors over supported catalysts. There are also some slower liquid phase reactions. U.S. Pat. No. 6,586,636, issued Jul. 1, 2003, introduced a process for preparation of unsaturated aldehydes from straight chain C₃, C₄ or C₆ vaporized aldehydes on a catalyst at 175° C. to 350° C. U.S. Pat. No. 6,552,232, issued Apr. 22, 2003, disclosed preparation of aldol products from aldehydes in ionic medium on supported base catalyst at −20° C. to 300° C., preferably 80° C. to 90° C. in a period of three hours at pressures of 1 atmosphere to 1000 atmospheres. This application makes use of neutral ionic liquid media and a basic catalyst where the ionic liquid medium is selected from 1-butyl-3-methyl imidazolium, 1-butyl-2,3-dimethyl imidazolium, 1-butyl-pyridinium, pyridinium or imidazolium species, and derivatives thereof, BF₄ ion and PF₆ ion species as well as basic catalyst comprising hydroxide species. U.S. Pat. No. 4,090,986, issued Jul. 18, 2000, discussed formation of esters from C₉ alcohols formed from a C₆ aldehyde and propanal by an aldol process producing>35% product then hydrogenating the unsaturated aldehyde to a saturated Cg aldehyde. U.S. Pat. No. 4,017,537, issued Apr. 12, 1977, taught preparation of aldol products from aldehydes with the aid of an esterfying agent at 150° C. to 280° C. There are also a number of records of prior art disclosing application of supported transition metal catalysts for both formation of aldehydes and for their reaction to products at elevated temperatures and pressures by means of an aldol condensation process. These reactions appear to be both slow and of low conversion efficiency.

The discovery taught in this application is chemical conversion of aldehydes to a majority of products comprising hydroxyaldehydes and/or polyhydroxyaldehydes, and ketones to products comprising hydroxyketones and/or polyhydroxyketones of controlled carbon chain length using selected concentrations of soluble inorganic bases at ambient pressure for reactant contact times of ten minutes or less at temperatures in the −25° C. to 50° C. range.

It is an object of this invention, therefore, to provide chemical conversion of aldehydes to products comprising hydroxyaldehydes and/or polyhydroxyaldehydes, and ketones to products comprising hydroxyketones and/or polyhydroxyketones of controlled carbon chain length using selected concentrations of soluble inorganic bases at ambient pressure for reactant contact times of ten minutes or less at temperatures in the −25° C. to 50° C. temperature range.

It is another object of this invention to teach that acetaldehyde reactant produces products comprising 3-hydroxybutyraldehyde (C₄) using 0.0005 g/mL of soluble base, 3-hydroxybutyraldehyde and 3,5-dihydroxyhexeraldehyde (C₄ and C₆) using 0.004 g/mL of soluble inorganic base, C₆ to C₁₄ using 0.03 g/mL of soluble base, C₁₂ to C₃₂ using 0.05 g/mL of soluble base and up to C₂₅₀ using 0.3 g/mL of soluble base for the soluble portion of inorganic bases comprising sodium, lithium, potassium, rubidium, cesium, magnesium, calcium, barium, strontium hydroxides, carbonates, bicarbonates and/or phosphates. Other objects of this invention will be apparent from the detailed description thereof that follows, and from the claims.

### SUMMARY OF THE INVENTION

This invention relates to ambient pressure chemical conversion of aldehydes to hydroxyaldehydes and/or polyhydroxyaldehydes, and ketones to hydroxyketones and/or polyhydroxyketones in an aqueous liquid phase by an aldol condensation process where carbon-carbon chain length of products is controlled by the concentration of a soluble base present. Reactions have been reproducibly conducted in a temperature range of −25° C. to 50° C. by rapid mixing of the reactant with a base solution at fixed concentrations. Reactions are completed in one to two minutes for most conditions, and in less than ten minutes for quite dilute base, following reactant addition.

### DETAILED DESCRIPTION OF THE INVENTION

Chemical conversion of aldehydes to products comprising hydroxyaldehydes and/or polyhydroxyaldehydes, and ketones to products comprising hydroxyketones and/or polyhydroxyketones of controlled carbon-carbon chain length using selected concentrations of soluble inorganic bases at ambient pressure for product formation completed in ten minutes or less. Acetaldehyde produced products comprising 3-hydroxybutyraldehyde (C₄) using 0.0005 g/mL of soluble base, 3-hydroxybutyraldehyde and 3,5-dihydroxyhexeraldehyde (C₄ and C₆) using 0.004 g/mL of soluble inorganic base, C₆ to C₁₄ using 0.03 g/mL of soluble base, C₁₂ to C₃₂ using 0.05 g/mL of soluble base and up to C₂₅₀ using 0.3 g/mL of soluble base for the soluble portion of inorganic bases comprising sodium, lithium, potassium, rubidium, cesium, magnesium, calcium, barium, strontium, carbonates, bicarbonates, phosphates and/or borates at ambient pressure for reactant contact times of ten minutes or less at temperatures in the −25° C. to 50° C. range.

A series of aldol reactions was conducted in aqueous liquid phase at 0° C. to 10° C. by varying the concentration of sodium hydroxide. Hydroxide concentration was adjusted to control the degree of oligomerization such that aldol formation was rapid and exothermic. A number of examples of specific product formation from acetaldehyde are presented here.

### EXAMPLE 1

Acetaldehyde, 11.0 grams, was injected at a rate of approximately 5 mL per minute into 10 mL of a rapidly stirred water solution containing 3.0 grams of sodium hydroxide cooled to a temperature of 5° C. to 10° C. A deep red suspension formed and the reaction was pH neutralized two minutes after the addition was completed resulting in formation of a red oil comprising a range of oligomeric products up to C₂₅₀.

### EXAMPLE 2

Into 10 mL of a rapidly stirred water solution containing 0.50 gram of sodium hydroxide cooled to a temperature of 5° C. to 10° C. was injected 11.8 grams acetaldehyde at a rate of approximately 6 mL per minute. A bright yellow dispersion formed and the reaction was pH neutralized two minutes after the addition was completed resulting in formation of a golden yellow oil comprising a distribution of products from C₁₂ to C₃₂.

### EXAMPLE 3

Into 10 mL of a rapidly stirred water solution containing 0.30 gram of sodium hydroxide cooled to a temperature of 5° C. to 10° C. was injected 4.4 grams acetaldehyde at a rate of approximately 5 mL per minute. A yellow dispersion formed and the reaction was pH neutralized two minutes after the addition was completed resulting in formation of a bright yellow oil comprising a distribution of products from C₆ to C₁₄.

### EXAMPLE 4

Into 10 mL of a rapidly stirred water solution containing 0.041 gram of sodium hydroxide cooled to a temperature of 5° C. to 10° C. was injected 6.2 grams acetaldehyde at a rate of approximately 2 mL per minute. A clear solution formed and the reaction was pH neutralized two minutes after the addition was completed resulting in formation of colorless organic products comprising C₄ and C₆.

### EXAMPLE 5

Into 10 mL of a rapidly stirred water solution containing 0.005 gram of sodium hydroxide cooled to a temperature of 5° C. to 10° C. was injected 9.2 grams acetaldehyde at a rate of approximately 6 mL per minute. A clear solution formed and the reaction was pH neutralized ten minutes after the addition was completed resulting in formation of colorless organic products comprising C₄.

### EXAMPLE 6

Into 10 mL of a rapidly stirred water solution containing 0.090 gram of sodium hydroxide cooled to a temperature of 5° C. to 10° C. was injected 3.4 grams acetaldehyde at a rate of approximately 6 mL per minute. A clear solution formed and the reaction was pH neutralized two minutes after the addition was completed resulting in formation of colorless organic products comprising C₄ to C₈.

1. Water soluble or dispersible liquid and solid non-glucose aldehydes have been chemically converted to greater than 50 percent products comprising selected molecular weight hydroxyaldehydes and/or polyhydroxyaldehydes using inorganic bases in a specific concentration range comprising 0.0005 g/L to 1.0 g/L, producing a pH greater than 9, at ambient pressure and temperatures in the −25° C. to 50° C. range.

2. Water soluble or dispersible liquid and solid non-glucose alphatic aldehydes have been chemically converted to greater than 50 percent products comprising selected molecular weight hydroxyaldehydes and/or polyhydroxyaldehydes using inorganic bases in a specific concentration range comprising 0.0005 g/L to 1.0 g/L, producing a pH greater than 9, at ambient pressure and temperatures in the −25° C. to 50° C. range.

3. Liquid and solid aldehydes comprising acetaldehyde have been chemically converted to greater than 50 percent products comprising a selected molecular weight of 3-hydroxybutyraldehyde (C₄) using a specific concentration of 0.0005 g/mL of base for the soluble portion of inorganic bases comprising sodium, lithium, potassium, rubidium, cesium, magnesium, calcium, barium and/or strontium hydroxides, carbonates, bicarbonates, phosphates and/or borates producing a pH greater than 9, at ambient pressure for reactant contact times of ten minutes or less.

4. Liquid and solid aldehydes comprising acetaldehyde have been chemically converted to greater than 50 percent products comprising selected molecular weights of 3-hydroxybutyraldehyde to 3,5-dihydroxyhexyralaldehyde (C₄ to C₆) using a specific concentration of 0.004 g/mL of base for the soluble portion of inorganic bases comprising sodium, lithium, potassium, rubidium, cesium, magnesium, calcium, barium and/or strontium hydroxides, carbonates, bicarbonates, phosphates and/or borates producing a pH greater than 9, at ambient pressure for reactant contact times of ten minutes or less.

5. Liquid and solid aldehydes comprising acetaldehyde have been chemically converted to greater than 50 percent products comprising selected molecular weights of C₆ to C₁₄ dihydroxyaldehydes to polyhydroxyaldehydes using a specific concentration of 0.03 g/mL of base for the soluble portion of inorganic bases comprising sodium, lithium, potassium, rubidium, cesium, magnesium, calcium, barium and/or strontium hydroxides, carbonates, bicarbonates, phosphates and/or borates producing a pH greater than 9, at ambient pressure for reactant contact times of ten minutes or less.

6. Liquid and solid aldehydes comprising acetaldehyde have been chemically converted to greater than 50 percent products comprising selected molecular weights of C₁₂ to C₃₂ pentahydroxyaldehydes to polyhydroxyaldehydes using a specific concentration of 0.05 g/mL of base for the soluble portion of inorganic bases comprising sodium, lithium, potassium, rubidium, cesium, magnesium, calcium, barium and/or strontium hydroxides, carbonates, bicarbonates, phosphates and/or borates producing a pH greater than 9, at ambient pressure for reactant contact times of ten minutes or less.

7. Liquid and solid aldehydes comprising acetaldehyde have been chemically converted to greater than 50 percent products comprising selected molecular weights of up to C₂₅₀ polyhydroxyaldehydes using a specific concentration of 0.3 g/mL of base for the soluble portion of inorganic bases comprising sodium, lithium, potassium, rubidium, cesium, magnesium, calcium, barium and/or strontium hydroxides, carbonates, bicarbonates, phosphates and/or borates producing a pH greater than 9, at ambient pressure for reactant contact times of ten minutes or less.

8. Liquid and solid aldehydes comprising acetaldehyde have been chemically converted to greater than 50 percent products comprising a selected molecular weight of 3-hydroxybutyraldehyde (C₄) using a specific concentration of 0.0005 g/mL of base for the soluble portion of inorganic bases comprising sodium, lithium, potassium, rubidium, cesium, magnesium, calcium, barium and/or strontium hydroxides, carbonates, bicarbonates, phosphates and/or borates producing a pH greater than 9, at ambient pressure for reactant contact times of ten minutes or less for product formation at temperatures in the −25° C. to 50° C. temperature range.

9. Liquid and solid aldehydes comprising acetaldehyde have been chemically converted to greater than 50 percent products comprising selected molecular weight of 3-hydroxybutyraldehyde to 3,5-dihydroxyhexyralaldehyde (C₄ to C₆) using a specific concentration of 0.004 mL of base for the soluble portion of inorganic bases comprising sodium, lithium, potassium, rubidium, cesium, magnesium, calcium, barium and/or strontium hydroxides, carbonates, bicarbonates, phosphates and/or borates producing a pH greater than 9, at ambient pressure for reactant contact times of ten minutes or less for product formation at temperatures in the −25° C. to 50° C. temperature range.

10. Liquid and solid aldehydes comprising acetaldehyde have been chemically converted to greater than 50 percent products comprising selected molecular weights of C₆ to C₁₄ dihydroxyaldehydes to polyhydroxyaldehydes using a specific concentration of 0.03 g/mL of base for the soluble portion of inorganic bases comprising sodium, lithium, potassium, rubidium, cesium, magnesium, calcium, barium and/or strontium hydroxides, carbonates, bicarbonates, phosphates and/or borates producing a pH greater than 9, at ambient pressure for reactant contact times of ten minutes or less for product formation at temperatures in the −25° C. to 50° C. temperature range.

11. Liquid and solid aldehydes comprising acetaldehyde have been chemically converted to greater than 50 percent products comprising selected molecular weights of C₁₂ to C₃₂ pentahydroxyaldehydes to polyhydroxyaldehydes using a specific concentration of 0.05 g/mL of base for the soluble portion of inorganic bases comprising sodium, lithium, potassium, rubidium, cesium, magnesium, calcium, barium and/or strontium hydroxides, carbonates, bicarbonates, phosphates and/or borates producing a pH greater than 9, at ambient pressure for reactant contact times of ten minutes or less for product formation at temperatures in the −25° C. to 50° C. temperature range.

12. Liquid and solid aldehydes comprising acetaldehyde have been chemically converted to greater than 50 percent products comprising selected molecular weights of up to C₂₅₀ polyhydroxyaldehydes using a specific concentration of 0.3 g/mL of base for the soluble portion of inorganic bases comprising sodium, lithium, potassium, rubidium, cesium, magnesium, calcium, barium and/or strontium hydroxides, carbonates, bicarbonates, phosphates and/or borates producing a pH greater than 9, at ambient pressure for reactant contact times of ten minutes or less for product formation at temperatures in the −25° C. to 50° C. temperature range.

13. Liquid and solid ketones comprising acetone have been chemically converted to greater than 50 percent products comprising selected molecular weights of hydroxyketones and/or polyhydroxyketones using solid inorganic bases producing a pH greater than 9, heated with a catalyst comprising cobalt tetrachlorocatechol in a sealed reactor at temperatures up to 150° C. for reactant times of less than one hour.

14. (canceled)
